(19) United States
(12) Patent Application Publication
(10) Pub. No.: US 2023/0313083 A1
(43) Pub. Date: Oct. 5, 2023

(54) CLEAN-IN-PLACE METHODS FOR FERMENTATION OPERATIONS

(71) Applicant: ECOLAB USA INC., St. Paul, MN (US)

(72) Inventors: Chris NAGEL, St. Paul, MN (US); Michael D. VANDER VORSTE, Sioux Falls, SD (US); Jennifer STOKES, Rosemount, MN (US); Junzhong LI, Eagan, MN (US); Jesse MURPHY, Apple Valley, MN (US); Mark Raymond ALTIER, Mendota Heights, MN (US); Zach BABCOCK, Nashville, TN (US); Christina RAMSUMAIR, Lakeville, MN (US)

(21) Appl. No.: 18/295,481

(22) Filed: Apr. 4, 2023

Related U.S. Application Data
(60) Provisional application No. 63/327,262, filed on Apr. 4, 2022.

(51) Int. Cl. C11D 11/00

(52) U.S. Cl. CPC C11D 11/0041

(57) ABSTRACT

Clean-in-place methods applicable to improve production of fermentation operations are disclosed herein to monitor, report, and control the cleaning reagents applied, residual components thereof, and clean-in-place conditions within levels that are compatible with fermentation processes, without limiting the effectiveness of the clean-in-place protocol with respect to cleaning or sanitizing the fermentation system.

### CROSS-REFERENCE TO RELATED APPLICATION(S)

This application claims priority to U.S. application Ser. No. 63/327,262 filed on Apr. 4, 2022, the disclosure of which is hereby incorporated by reference in its entirety.

### BACKGROUND

Industrial fermentation operations are applicable to food manufacturing processes, beer and wine making, biotech processes, and biofuel production where enzymes and yeasts convert starches and sugars from plants into alcohols. The alcohol yield largely depends on the activity and health of yeast and/or relevant enzymes employed throughout the processes. The presence of plant matter, yeasts, starches, sugars, and the other materials within the fermentation operation cause soil build up, an opportunity for growth of unwanted bacteria that can compete with the yeast in a fermentation process, and generation of unwanted byproducts such as acetic acid and lactic acid. The unwanted soil and byproducts are periodically removed by cleaning.

Clean-in-place (CIP) systems have been developed to allow cleaning in fermentation operations in between fermentation batches without the need to dismantle equipment and interrupt production beyond the length of time required for the clean-in-place program. Clean-in-place systems are thereby able to minimize downtime of fermentation operations and maintain a sanitary condition for the fermentation operation over many subsequent fermentations. In addition to cleaning the fermentation tanks, the clean-in-place systems also clean other equipment within the fermentation operation, including mash trains, fermenters, piping, yeast propagation tanks, and other tanks, pipes, pumps, heat exchangers, evaporators, and equipment.

Clean-in-place methods apply cleaning or sanitizing chemicals to remove soils and unwanted bacteria. But running a clean-in-place program in a fermentation operation presents certain challenges. For example, industrial biofuel fermentation processes can be limited or closed with respect to waste disposal allowing only animal feed, ethanol, syrup, corn oil, and steam from the fermentation process as outputs with all other chemicals being recycled to subsequent fermentation batches. Used clean-in-place solutions also cannot be discarded through municipal systems and are recycled back through the fermentation process. The used clean-in-place solutions can negatively affect the biology and chemistry of subsequent fermentation.

Clean-in-place methods to effectively clean or sanitize fermentation systems without interfering with yeast or enzyme health and productivity in subsequent fermentation runs are therefore desired. It is against this background that the present disclosure is made.

### SUMMARY

Methods for managing the cleaning-in-place of a fermentation operation while improving fermentation efficiency are disclosed herein. In certain aspects, the methods comprise monitoring a fermentation parameter, determining if the fermentation parameter is within an acceptable range, and modifying a clean-in-place program to bring the fermentation parameter within the acceptable range. In other aspects, methods for managing the clean-in-place systems can comprise monitoring the sodium ion concentration in at least one fermentation tank, determining if the sodium ion concentration is within an acceptable range, and modifying a clean-in-place program to bring the sodium ion concentration within the acceptable range. A method of managing the clean-in-place of a fermentation operation while improving fermentation efficiency also can comprise monitoring a fermentation parameter, recording the fermentation parameter, and determining if the fermentation parameter is within an acceptable range.

Methods of disposing of used sodium-containing clean-in-place solutions in a fermentation operation are also disclosed herein, and can comprise transferring a first cleaning composition comprising used sodium-containing clean-in-place solutions from a clean-in-place tank to a holding tank in fluid communication with the clean-in-place tank, measuring the sodium ion concentration in a plurality of fermentation tanks, and adding a portion of the first cleaning composition to the plurality of fermentation tanks wherein the sodium ion concentration within a fermentation tank is within +/−25% of the sodium ion concentration in the other fermentation tanks. Similar methods can be used with fermentation parameters other than sodium ion concentration.

In other aspects, use of a dedicated holding tank can be avoided. Methods of disposing of used sodium-containing clean-in-place solutions in a fermentation operation disclosed herein can comprise measuring the sodium ion concentration in a plurality of fermentation tanks, and transferring a first cleaning composition comprising used sodium-containing clean-in-place solutions from a clean-in-place tank to the plurality of fermentation tanks, wherein the sodium ion concentration in each fermentation tank does not exceed 100 ppm or wherein the sodium ion concentration in each fermentation tank is within +/−25% of the sodium ion concentration in the other fermentation tanks. Similar methods can be used with fermentation parameters other than sodium ion concentration.

In some aspects, methods disclosed herein can comprise measuring the sodium ion concentration in a plurality of fermentation tanks comprising one sacrificial fermentation tank and a plurality of preserved fermentation tanks, and transferring a first cleaning composition comprising used sodium-containing clean-in-place solutions from a clean-in-place tank to the sacrificial fermentation tank, wherein the sodium ion concentration in each preserved fermentation tank does not exceed 100 ppm.

In some aspects, used clean-in-place solutions may be discarded by blending the used clean-in-place solution into other portions of the fermentation system, bypassing the fermentation tanks altogether and the resulting elevated sodium concentrations. In certain aspects, the discarded clean-in-place solutions therefore can be distributed throughout the post-fermentation processes before being partially returned to the fermentation tanks by recycled waters generated during post-fermentation processing. This approach allows post-fermentation processing to better distribute sodium received at the fermentation tanks that may otherwise be delivered in bolus form if the clean-in-place solution was discarded at one time, and also allows sodium to exit the system partially by solid waste streams (e.g., dried distillers grains and solubles (DDGS) animal feed or syrup), without negatively affecting the post-fermentation processing of the biofuel product. For instance, an amount of the used clean-in-place solutions may be blended into the post-fermentation product lines (e.g., the beerwell or transfer lines leading to the beerwell) following each fermentation run such that the total amount of clean-in-place solution is blended into the process without overloading sodium concentrations at any single point in the fermentation system. In certain aspects, the used clean-in-place solution can be blended into the fermentation product stream. In other aspects, used clean-in-place solution can be blended into the fermentation product line or downstream fermentation processing equipment in a continuous or periodic manner during the fermentation process.

### BRIEF DESCRIPTION OF THE DRAWINGS

FIG. 1 depicts a schematic diagram of a fermentation system having a clean-in-place system incorporated within.

FIG. 2 depicts a schematic diagram showing additional flow paths leaving the clean-in-place systems into the fermentation system.

FIG. 3 depicts a schematic diagram showing another view of the fermentation system with the outlet for the carbon dioxide and water vapor.

FIG. 4 depicts a sample report generated by a clean-in-place controller.

FIG. 5 is a chart detailing results from yeast growth analysis in various concentrations of sodium.

FIG. 6 is a chart detailing results from Amylase Blend #1 performance under various salt concentrations.

FIG. 7 is a chart detailing results from Amylase Blend #1 performance under various salt concentrations over 30 minutes.

FIG. 8 is a chart detailing results from Amylase Blend #1 performance under various sodium concentrations over 30 minutes.

FIG. 9 is a chart detailing results from Amylase Blend #2 performance under various salt concentrations.

FIG. 10 is a chart detailing results from Amylase Blend #2 performance under various salt concentrations over 120 minutes.

FIG. 11 is a chart detailing results from Amylase Blend #2 performance under various sodium concentrations over 120 minutes.

### DETAILED DESCRIPTION

Clean-in-place cleaning in fermentation operations is challenging. Fermentation operations typically rely on yeast to convert sugars into ethanol. Enzymes also may be included within the fermentation reaction to convert complex starches, as can be present in corn mash for instance, into simple sugars available for yeast to produce the desired ethanol product. Enzymes also may be added to convert proteins into amino acid nutrients to supply yeast propagation. Corn mash fermentation operations therefore can produce complex mixtures of organic and inorganic ingredients that can leave behind residual soils within the operating equipment. These soils include starches, sugars, proteins, fats, and mineral deposits that accumulate on the inside of the equipment and need to be removed. Additionally, the natural conversion of corn mash into ethanol means that the corn mash soil evolves over time. The dynamic nature of the residual corn mash soil makes it more difficult to remove than a static soil. Finally, fermentation operations release carbon dioxide into the solutions and vapor space within the equipment. This carbon dioxide may react negatively chemically with the cleaning and sanitizing agents.

Industrial fermentation operations for making ethanol are constrained by strict standards for waste disposal. For example, most biofuel fermentation operations are not permitted to send any waste to the drain, and therefore the only way for material to leave an ethanol plant is through steam, product streams (e.g., ethanol), or by-product streams (e.g., fermentation solids that are converted into another product such as animal feed, corn oil, or syrup). Clean-in-place solutions incorporated within the fermentation operations are also subject to waste stream restrictions and can accumulate within the fermentation system if not carefully managed, resulting in negative effect on the intended fermentation processes.

Moreover, clean-in-place solutions can introduce significant quantities of yeast-stressing analytes into the system that can be difficult to remove from the equipment after cleaning. For instance, sodium hydroxide is commonly employed within clean-in-place programs as a cost-efficient and effective cleaning agent. As disclosed herein, it was surprisingly found that even very low concentrations of sodium can have a significant negative effect on yeast productivity and performance.

Clean-in-place methods for limiting sodium concentrations in fermentation processes are disclosed herein to improve productivity of fermentation operations without sacrificing cleaning ability. Methods disclosed herein comprise monitoring, reporting, and ultimately controlling the amount of sodium and other reagents, by-products, and conditions applied during clean-in-place programs, and unexpectedly found to significantly increase the ethanol yield during subsequent fermentation runs while achieving the desired cleaning or sanitizing objectives. Broadly, methods disclosed herein can be conducted by fermentation systems comprising relevant sensors, processors, and reporting interfaces as described in further detail below.

### Clean-in-Place Operations

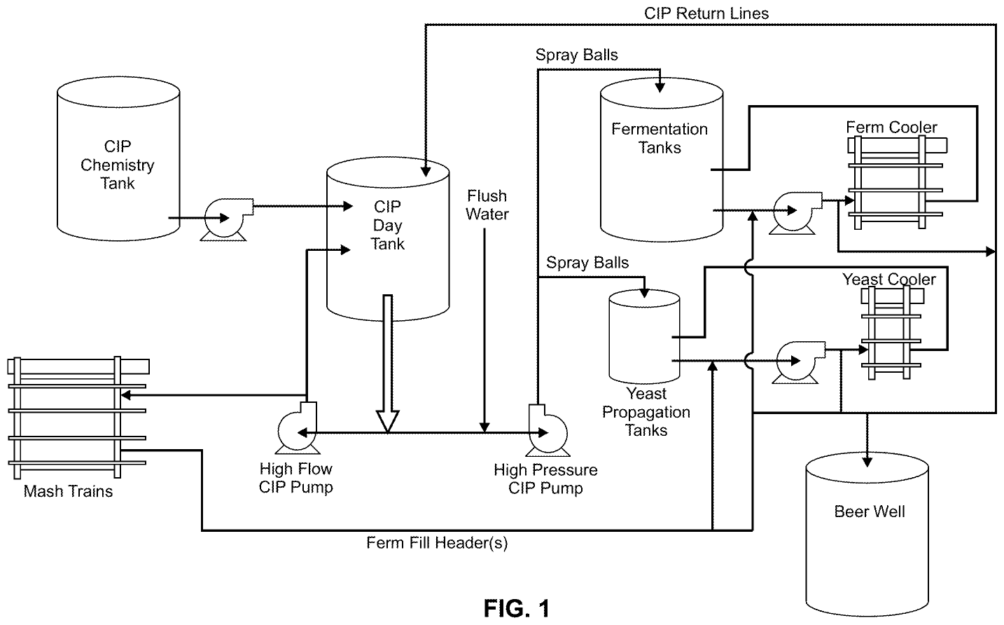

In fermentation operations, the fermentation system includes a series of tanks, pipes, pumps, heat exchangers, evaporators, dryers, and other equipment required to carry out the fermentation process from the delivery of the plant material (e.g., corn, barley, milo, etc.) to the generation of the end products (e.g., ethanol, DDGS, syrup, corn oil, steam). Some of the equipment used in the fermentation process is shown in FIGS. 1-3. The clean-in-place system is separate from the fermentation system and includes tanks for storing the clean-in-place solutions. Fermentation operations may be conducted in the fermentation tanks in batches. Periodically, the fermentation tanks are cleaned in between fermentation batches by running a cleaning cycle. During cleaning, cleaning chemicals from the clean-in-place system are pumped to the fermentation tanks and circulated for a period of time of about 10 minutes, about 20 minutes, about 30 minutes, about 45 minutes, about 60 minutes, about 90 minutes, or about 120 minutes, about 10-120 minutes, about 10-90 minutes, about 10-60 minutes, or about 10-30 minutes. The clean-in-place solutions are used at temperatures of about 20° C. to about 120° C., about 30° C. to about 100° C., or about 50° C. to about 90° C. Once the cleaning cycle is complete, the chemicals are pumped back to the recovery tank to wait and be used in the next cleaning. Sodium hydroxide is a commonly used clean-in-place chemical because it is a powerful cleaner and inexpensive. The cleaning cycle may optionally be followed by a water rinse. The cleaning cycle may also optionally be followed by a sanitizing cycle using an antimicrobial agent. Other portions of the fermentation system are also cleaned in a similar manner.

After being used for multiple clean-in-place operations, the clean-in-place solutions becomes too dirty or are chemically converted into other, less effective, chemicals. In this way, the clean-in-place solutions can become “spent” and need to be disposed of and replaced with fresh chemicals. One way to dispose of spent cleaning chemicals is to neutralize it and send it to the drain. In operations where a plant is not permitted to send anything to the drain, the chemicals need to be disposed of in other ways. Such methods for disposing of clean-in-place solutions are disclosed herein.

### Clean-in-Place Monitoring

Methods of managing cleaning-in-place of fermentation operations are disclosed herein, and generally include monitoring a fermentation parameter, determining if the fermentation parameter is within an acceptable range, and modifying a clean-in-place program to bring the fermentation parameter within the acceptable range. In this manner, it is contemplated that the fermentation operations may be improved by more careful control of fermentation parameters during the clean-in-place procedures, to ensure efficient ethanol production in subsequent fermentation processes.

Fermentation parameters relevant to be monitored in clean-in-place methods disclosed herein are not limited to any particular parameter or condition and can be any that improve the performance of subsequent fermentation operations. In certain aspects, monitoring a fermentation parameter can comprise monitoring an ion concentration, such as pH, sodium, potassium, calcium, magnesium, or combinations thereof. Monitoring a fermentation parameter also can comprise monitoring concentrations of fermentation reagents and intermediates, including but not limited to starch, simple and complex sugars, ethanol, ammonia, urea, sulfuric acid, acetic acid, lactic acid, and combinations thereof. Fermentation parameters also can include conditions of the fermentation processes such as viscosity, temperature, and pressure. Viscosity measurements after the slurry tank are an indicator of enzyme activity. Enzymes start converting the starches in the corn mash into sugars in the slurry tank. As this happens, the viscosity of the corn mash decreases. If the enzymes are not functioning sufficiently, the viscosity will not decrease as it is expected to. Measuring viscosity is thus an indicator of enzyme activity and health. Fermentation parameters may also include direct measurements of yeast health and enzyme activity, such as by a metabolic rate indicator described within U.S. Pat. No. 9,631,219, or by monitoring yeast budding, measuring overall yeast counts, and measuring intermediate sugar products as an indicator of enzyme activity. Fermentation parameters may also monitor the reagents in the system including antimicrobial concentration, antibiotic concentration, alkalinity concentration, and the like.

In certain aspects, sodium ion concentration can be maintained within an acceptable range to prevent sodium from being retained or accumulating within the fermentation system. In certain aspects, an acceptable range for sodium can be less than 120 ppm, less than 100 ppm, less than 80 ppm, or less than 60 ppm. Background sodium levels can be present within the corn mash and do not interfere with the fermentation productivity. In certain aspects, an acceptable range of sodium ions can be at least 10 ppm, at least 20 ppm, or at least 35 ppm. Acceptable ranges of sodium concentrations can include from 10 to 100 ppm, from 0 to 80 ppm, from 35 to 60 ppm, from 20 to 80, or from 20 to 50 ppm sodium.

Acceptable sodium levels may also be determined relative to other fermentation parameters disclosed herein. For instance, without being bound by theory, it is contemplated that adding ions to the fermentation system that compete with sodium may limit or reduce the amount of sodium retained following clean-in-place methods disclosed herein. Potassium ions for instance may provide a suitable blocking effect for sodium in high concentrations. In certain aspects, acceptable concentrations of potassium may be at least 500 ppm, at least 1000 ppm, at least 1500 ppm, at least 2500 ppm, at least 3500 ppm, at least 5000 ppm, or in a range from 500 to 5000 ppm or from 1000 to 2500 ppm. Alternatively, a ratio of potassium to sodium may be in a range from 10:1 to 1000:1, from 10:1 to 100:1, from 30:1 to 500:1, or from 30:1 to 100:1.

Fermentation parameters contemplated herein also include compounds that indicate the presence and proliferation of bacteria within the fermentation system, such as acetic acid and lactic acid. In certain aspects, acceptable levels of acetic acid can be less than 0.05%, or less than 0.1%. Acceptable levels of lactic acid can be less than 0.1%, less than 0.3%, less than 0.5%, less than 0.8%, or less than 1.0%. Levels of bacteria products and yeast byproducts can been correlated to the presence of bacteria within the fermentation system, and accordingly provide an indication that the clean-in-place program is working (or not working). If such byproducts are found to exceed acceptable ranges, that indicates an increase in the amount of applied sanitizing agents in order to kill off excess bacteria present within the system.

Lactic acid also may be produced as a byproduct of enzymes commonly added to fermentation processes. Protease, for instance, will generate lactic acid. Accordingly, monitoring lactic acid levels can be conducted in conjunction with and with consideration to the amount of protease or other enzymes present in the fermentation mixture.

It is also contemplated that controlling sodium levels to be within the ranges disclosed herein may improve yeast health, resulting in a much higher tolerance to lactic acid, acetic acid, or low pH conditions generally during the fermentation process. In turn, more robust yeast will not demand stronger clean-in-place solutions that can often impair yeast health in subsequent fermentation batches and cause a runaway negative effect on the overall health of the fermentation system.

However, it is surprisingly determined that yeast health may be more affected in the presence of sodium outside the acceptable ranges indicated herein, as compared to these conventional markers for yeast health and presence of bacteria within the fermentation system. Maintaining the sodium levels within an acceptable range with priority over conventional markers may ultimately lead to higher productivity, and less competition from bacteria present in the fermentation system.

Acceptable ranges of conditions may also be monitored and maintained throughout the clean-in-place and fermentation systems. In certain aspects, the pH can be maintained within a range acceptable for yeast health, e.g., from 4 to 8, from 4 to 6.5, from 4.5 to 6.5, from 4.5 to 5.5, or from 4.5 to 5. The acceptable range for pH may also be defined by the pKa of a component of the fermentation, such as lactic acid and acetic acid, each of which may have a corrosive and toxic affect in their acid form. Temperature may also be monitored and maintained within acceptable ranges for yeast health and productivity. In certain aspects, temperature can be maintained within a range from 20 to 40° C., or from 25 to 35° C. Acceptable ranges of other markers of yeast health and productivity such as urea, ammonia, and ethanol, may also be retained within conventional ranges.

Sugar concentrations also may be monitored as contemplated herein, as yet another measure of yeast and enzyme health and productivity. Total sugars may be evaluated as the amount of sugar present in the fermentation mixture as including both simple sugars and complex starches. Acceptable range for total sugars as contemplated herein can be less than 2%, less than 1%, less than 0.8%, or less than 0.5%, or in a range from 0.1% to 0.5%, or from 0.1% to 1.0%. Individual sugars can also be monitored as enzymes convert starches into simpler forms of sugar. Accumulation of certain sugar intermediates may be an indication that the enzymes are not able to effectively break down starches into the simplest sugar form.

Methods contemplated herein also can comprise monitoring more than one fermentation parameter disclosed herein, such as any fermentation parameter in combination with sodium ion concentrations at any point within the fermentation system. Monitoring fermentation parameters as described above can be achieved by any instrumentation appropriate to collect the relevant data, and through detection at any suitable point within the fermentation or clean-in-place system.

In certain aspects, sensors and probes for measuring a fermentation parameter can be placed in a tank within the clean-in-place system, e.g., a rinse tank, a cleaning concentrate tank, a recycle tank, or combinations thereof. The sensors and probes can also be placed within the fermentation system. Exemplary locations within the fermentation system include the bottom of the scrubber, the bottom of the side stripper, in the slurry tank, the liquefaction tank, one or more fermentation tanks, the beerwell tank, the backset line, the yeast propagation tank, before the drum dryer, before the syrup tank, or within any feed or return line in fluid communication with the clean-in-place or fermentation operation, such as is depicted generally in FIGS. 1-3.

In some embodiments, it may be beneficial to monitor one or more fermentation parameters at multiple locations within the fermentation system. Monitoring a fermentation parameter at multiple locations will allow an operator to observe how certain analytes are moving in or out of the system. Using sodium as an example, if the sodium ion concentration within the system becomes too high, the clean-in-place solutions may be switched in favor of cleaning chemicals that are lower in sodium. However, any sodium in the system still needs to work its way out of the system through animal feed, syrup, or other by-product. Placing sensors, for example, in the backset line, in the slurry tank, in the fermentation tanks, in the beerwell tank, and before the drum dryer or syrup tanks will allow an operator to observe how much sodium is leaving the system as animal feed or syrup, and how much is being recycled back into the system through the backset line.

Monitoring a fermentation parameter also can be conducted at any point during the fermentation process, for instance while running a clean-in-place program, after completing a clean-in-place program, during start-up of a fermentation batch, intermittently or throughout the fermentation batch, after a fermentation batch, or combinations thereof. Monitoring also may be conducted at any sampling speed appropriate to return sufficient information to the clean-in-place system and/or fermentation system to exert intended control over the processes.

The measuring of fermentation parameters can be accomplished using various sensors. For monitoring ions, ion-selective electrodes, Near Infrared Spectrometers (NIR), or HPLC can be used. pH probes and temperature sensors can be used to measure pH and temperature. Yeast health can be measured using a metabolic rate indicator such as the one described within U.S. Pat. No. 9,631,219. Yeast counts and yeast budding can be determined using a microscope.

Once a fermentation parameter value has been determined as described above, or otherwise, the value may be returned to a processor within the clean-in-place system or fermentation control system to determine whether the fermentation parameter is within an acceptable range. Methods disclosed herein can comprise determining whether the fermentation parameter obtained as described above is within an acceptable range. In certain aspects, the fermentation parameter can be obtained at a sensor within the fermentation system, e.g., a sodium concentration sensor within a fermentation tank, and the associated fermentation parameter value returned to a controller within the clean-in-place system or fermentation system.

Methods disclosed herein can further comprise modifying the clean-in-place program to bring the fermentation parameter within the acceptable range, if it is determined that the fermentation parameter is outside the acceptable range at any point during monitoring. In certain aspects, modifying the clean-in-place program can comprise providing additional water input to the clean-in-place supply chemicals to reduce the concentration of a fermentation parameter to the acceptable range. Modifying the clean-in-place program also can include redirecting flow from a single fermentation tank to multiple fermentation tanks within the fermentation system to equalize the fermentation parameter across the multiple fermentation tanks. For instance, modifying the clean-in-place program can comprise adjusting the fermentation parameter to be within +/−30%, within +/−25%, or within +/−10% of the value across each fermentation tank within the fermentation system.

In other aspects, modifying the clean-in-place program can include adding a supplement to the clean-in-place supply chemicals or directly to the fermentation system to increase a fermentation parameter value, or alternatively to react with and neutralize a fermentation parameter. For instance, an alkaline substance may be added to the clean-in-place solutions to neutralize acid species in the fermentation tanks and maintain a pH within a determined acceptable range. Further still, modifying the clean-in-place program can comprise replacing the clean-in-place solutions, either partially or entirely. For instance, clean-in-place solutions can be replaced with clean-in-place solutions having a reduced sodium ion concentration, or a sodium-free clean-in-place solutions. In other aspects, the clean-in-place solutions may be replaced with chemicals having a pH less than 8, for instance where the pH of the clean-in-place program has exceeded an alkaline pH limit.

In other aspects, modifying the clean-in-place program can include a full emptying of the clean-in-place tank into a different tank (e.g., the beerwell or a waste clean-in-place tank). This is useful in operations where the clean-in-place solution can be disposed of. In other aspects, modifying the clean-in-place program can include bleeding or dosing off portions of the clean-in-place tank into a different tank (e.g., the beerwell or waste clean-in-place tank).

In some embodiments, the parameters of the beerwell tank are monitored and maintained at a constant concentration. For example, the sodium ion concentration can be monitored with a sensor and kept at a concentration of less than 100 ppm sodium ion, from 0 to about 1000 ppm sodium ion, from about 10 to about 500 ppm sodium ion, from about 10 to about 100 ppm sodium ion, from about 10 to about 50 ppm sodium ion, from about 50 to about 100 ppm sodium ion, from about 100 to about 500 ppm sodium ion, or from 0 to about 50 ppm sodium ion. Similar concentrations can also be used for other ions such as potassium, and calcium, and compounds such as nitrates or sulfates in the beerwell. As an analyte concentration in the beerwell goes below the desired concentration range, used clean-in-place composition can be added to the beerwell. If an analyte concentration in the beerwell goes too high, the clean-in-place composition can be discarded.

Surprisingly, methods disclosed herein have shown the ability to improve characteristics of subsequent fermentation processes, particularly relevant to conventional clean-in-place methods. In certain aspects, modifying the clean-in-place program as disclosed herein may result in improved yeast efficiency. For instance, fermentation operations comprising yeast can have a yeast lag time where production of ethanol is reduced at the initial stages. When the fermentation parameters are outside of the acceptable ranges, the yeast lag time may be on the order of 3 to 15 hours. Modifying the clean-in-place program as disclosed herein to bring the fermentation parameters within an acceptable range may shorten the lag time in yeast activity by 5%, 10%, or 20% compared to conventional clean-in-place methods.

More generally, modifying the clean-in-place program as disclosed herein can result in an increase in ethanol production, for instance due to an increased enzyme efficiency following a yeast lag time, a shortened yeast lag time, or both. In some embodiments, ethanol production can be increased by 0.5%, 1%, 2%, 3%, 4%, 5%, 6%, 7%, 8%, 9%, or 10% as a result of the methods disclosed herein. Alternatively, or additionally, modifying the clean-in-place program as disclosed herein may result in a reduction in components within the fermentation operation that have a negative effect on production. For instance, bacteria that compete with the yeast under fermentation conditions can consume resources during fermentation such as sugar, and thereby limit resources available for yeast. Such interference can introduce alternative and unwanted products into the fermentation product stream, including lactic acid and acetic acid bacteria byproducts. Thus, in certain aspects, the clean-in-place program can result in lower concentrations of acetic-acid generating bacteria, lactic-acid generating bacteria, or both. Modifying the clean-in-place program can result in lower acetic acid and lactic acid in the fermentation mixture and product streams.

### Sodium Ion Monitoring

Sodium concentrations within the fermentation system may significantly impair yeast activity during subsequent fermentation operations, particularly during yeast propagation and fermentation startup. Clean-in-place solutions are one contributor to the sodium ion concentration within the fermentation system. Sodium concentrations in clean-in-place solutions and the recycled waters within the fermentation system can increase over time where they cannot be removed through solid waste used for feed. Thus, while sodium hydroxide remains an effective and cost-efficient agent for clean-in-place programs, use of sodium hydroxide within industrial scale processes that operate as closed systems or systems where waste streams are limited can cause sodium concentrations to increase following repeated runs of a clean-in-place program that employs sodium hydroxide (or equivalent sodium-based chemicals).

As discussed above, methods for monitoring fermentation parameters such as sodium ion concentrations within such fermentation operations are contemplated herein and generally can comprise monitoring the sodium ion concentration in at least one fermentation tank, determining if the sodium ion concentration is within an acceptable range, and modifying a clean-in-place program to bring the sodium ion concentration within the acceptable range. In certain aspects, modifying the clean-in-place program can comprise changing the concentration of a clean-in-place solution, adding a supplement to the clean-in-place program or the fermentation system, replacing a clean-in-place solution within the clean-in-place system, controlling the discharge location of spent clean-in-place solutions, decisions about cleaning frequency, or combinations thereof. In certain aspects, replacing the clean-in-place solution can comprise replacing the clean-in-place solutions with reduced-sodium or sodium-free chemicals. In other aspects, replacing the clean-in-place solutions can comprise replacing the clean-in-place solutions with alternative clean-in-place solutions having a pH less than 8, a pH less than 7, a pH less than 6, a pH less than 5, a pH less than 4, or a pH from 4 to 8, 4 to 7, or 4 to 6.

### Monitoring and Reporting

Methods for monitoring and reporting fermentation parameters are also applicable to clean-in-place methods and fermentation operations disclosed throughout this disclosure. Generally, methods of managing a clean-in-place program of a fermentation operation are disclosed while improving fermentation efficiency are disclosed herein. Methods for managing clean-in-place programs can comprise monitoring a fermentation parameter, recording the fermentation parameter, and determining if the fermentation parameter is within an acceptable range. Methods may further comprise modifying the clean-in-place program or fermentation system to bring the fermentation parameter within the acceptable range.

In certain aspects, recording the parameter can comprise paper or electronic recording. In certain aspects, the output from sensors placed throughout the fermentation operation may be reported to a controller or a central digital database and relied upon for further control of the fermentation operation, output to an interface, or a printed or digital report. Monitoring can be performed continuously, in real-time, or intermittently such as once a shift or once a fermentation batch, or at regular intervals such as every 5 minutes, every 10 minutes, every 15 minutes, every 30 minutes, every 60 minutes, every 90 minutes, every two hours, every three hours, every six hours, every 12 hours, every 24 hours, or every 48 hours.

Methods for monitoring may further comprise generating a report comprising data collected while monitoring the fermentation parameters. In certain aspects, reports can comprise fields for data related to date, time, location, site, sensor name, sensor ID number, fermentation tank, fermentation parameter, corrective action, ethanol yield, an overall yeast health score, an overall enzyme health score, or a combination thereof. Overall yeast health scores or overall enzyme health scores may be a composite of various parameters indicative of yeast and enzyme health including yeast concentration, yeast budding, ethanol yield, total sugar, sugar intermediates, or interfering analytes. Reports may be digital reports such as an email, mobile phone message, data file of a digital storage medium, part of an in-line instrument, or within a dedicated display of the clean-in-place system, controller, or fermentation system. Alternatively, paper reports may be generated and stored as records for retention. The data contained in the reports may trigger an alert notifying a user that a fermentation parameter is out of range. The alert may be stored on the report or reported as an email, mobile phone message, visual alert, audio alert, or color-coded scoring system on the report (e.g., red for out of the acceptable range, yellow for nearly in or out of the acceptable range, green for within the acceptable range). In certain aspects, the alert may be delivered at the physical location of the clean-in-place system or fermentation system, or at a remote monitoring location.

### Bleeding Off Spent Clean-in-Place Solutions

Methods for balancing or reducing the sodium that may accumulate within such closed fermentation operations are also disclosed herein.

In certain aspects, methods for disposing of used sodium-containing clean-in-place solutions in a fermentation operation can comprise transferring a cleaning composition comprising used sodium-containing clean-in-place solutions from a clean-in-place tank to a holding tank in fluid communication with the clean-in-place tank, measuring the sodium ion concentration in a plurality of fermentation tanks, and adding the used sodium-containing clean-in-place solutions to the plurality of fermentation tanks wherein the sodium ion concentration in the mixture does not exceed 100 ppm. In some embodiments, a portion of the clean-in-place solutions are added to each fermentation tank. That amount can be the volume of the clean-in-place solutions divided by the number of fermentation tanks, a percentage based on the number of fermentation tanks, or a percentage in the range of 5% to 25%, 5% to 15%, or 5% to 10% of the clean-in-place solutions that are added to each fermentation tank or into other areas of the fermentation process.

In such aspects, the clean-in-place tank may be a recovery tank (e.g., a CIP day tank or beer well as shown in FIG. 1) configured to receive the cleaning composition comprising used sodium-containing clean-in-place solutions from a clean-in-place return in fluid communication with the fermentation tank outlet stream, within a closed waste system. The cleaning composition can be transferred from the recovery tank to the holding tank, in part or in whole. An exemplary holding tank may be the beerwell tank in FIG. 1 or a separate holding tank dedicated to the clean-in-place system.

In some embodiments, the used sodium-containing clean-in-place solution is replaced with a clean-in-place composition that is more favorable to yeast or enzyme health. If such compositions include concentrations of components that negatively affect yeast or enzyme health, then such compositions (1) include such components within a parameter range that can be managed so that yeast activity is not negatively affected, or no such components, and (2) such compositions include components that are compliant with any restrictions on the further use of the cleaning composition, such as animal feed. If the cleaning composition is going to be incorporated into a waste stream that is further used as animal feed, the cleaning composition components must be considered to be food or feed additive compatible.

The Food, Drug, and Cosmetic Act provides a partial list of food additive materials at 21 C.F.R. parts 573 and 579. A partial listing of substances that are considered GRAS (generally recognized as safe) is found at 21 C.F.R. parts 582 and 584. The Official Publication of the Association of American Feed Control Officials (AAFCO) includes a list of approved animal food (feed) ingredients. Many of the ingredients in the AAFCO Official Publication are not approved food additives or considered GRAS but are nevertheless recognized by the FDA as approved for animal feed as long as there are no safety concerns. In some embodiments, suitability for direct, indirect, or incidental food contact is determined by the United States Food and Drug Administration. In certain embodiments, the clean-in-place compositions disclosed herein are made with food additive materials, feed additive materials, or materials that are suitable for direct, indirect, or incidental food contact.

In certain aspects, the cleaning composition can be a reduced-sodium or sodium-free analog of the sodium-containing cleaning composition. Exemplary sodium ion replacements include potassium, aluminum, calcium, magnesium, and ammonium.

In other aspects, the cleaning composition can comprise an enzyme such as a protease, amylase, carbohydrase, and lipase. In one embodiment, an enzyme cleaner concentrate comprises 0-2 wt. % of an enzyme, a buffer to adjust the pH to a range of about 7 to about 11, or about 8 to about 10, or about 9 to about 10. When used in a clean-in-place process, the composition is dosed into the clean-in-place system so that the enzyme concentration within the clean-in-place process is about 0 to about 200 ppm.

In still further aspects, the cleaning composition can comprise a buffer used to adjust the sodium ion concentration of the mixture without adjusting the pH. The buffer may be either a sodium-free buffer, or a sodium-containing buffer.

In certain aspects, the cleaning composition can comprise an acid, for instance to adjust the observed sodium concentration within the mixture by adjusting the pH up or down as needed.

Methods for limiting the sodium in cleaning compositions for clean-in-place solutions are also contemplated that do not involve a dedicated holding tank. For instance, the sodium-containing compositions as described above may be directly transferred to one or more fermentation tanks such that the upper limit of sodium ion concentration is not exceeded during the clean-in-place program. As above, such methods can include measuring the sodium ion concentration in a plurality of fermentation tanks, and modifying the clean-in-place program accordingly to ensure that the sodium concentration does not exceed 100 ppm in any of the fermentation tanks.

Alternatively, one of the plurality of fermentation tanks may be used as a holding tank, and therefore act as a sacrificial fermentation tank to accept cleaning compositions comprising a sodium concentration in excess of the acceptable ranges as disclosed above. In this manner, the remainder of the fermentation tanks can be preserved in a low-sodium condition within the acceptable range. Such approach may be particularly attractive in fermentation operations comprising a larger number of tanks such that the ratio of preserved fermentation tanks to a lone sacrificial fermentation tank approaches 1. In certain aspects, the plurality of fermentation tanks can be in a range from 2 to 50, from 3 to 20, from 5 to 15, or from 2 to 10.

### Blending Fermentation Mixtures to Preserve Fermentation Conditions

Continuing from methods described above, it is further contemplated that fermentation parameters may be maintained within acceptable levels across multiple fermentation tanks by actively blending the fermentation mixtures within a plurality of fermentation tanks. Methods for managing clean-in-place of fermentation operations disclosed herein may be applied to improve fermentation efficiency and may comprise monitoring a fermentation parameter in a plurality of fermentation tanks comprising corn mash, determining if the fermentation parameter is within an acceptable range for each fermentation tank, and blending corn mash between fermentation tanks so that the fermentation parameter for each fermentation tank is within +/−10%, within +/−25%, or within +/−30%. For instance, each of a given plurality of fermentation tanks each may comprise a sugar ion concentration differing across a range of the fermentation tanks. Blending corn mash according to additional fermentation parameters as described herein is also contemplated, and can comprise sodium ion concentration, potassium ion concentration, acetic acid concentration, lactic acid concentration, urea concentration, ammonia concentration, sugar concentration, ethanol concentration, temperature, pH, and combinations thereof.

It is also contemplated herein that consecutive balancing steps may be conducted to balance a plurality of fermentation parameters within each fermentation tank. In this manner, the composition of each fermentation tank can be monitored and adjusted within an acceptable range for any number of fermentation parameters without requiring independent additives. As the biologic processes continue, production within each of the fermentation tanks can be normalized toward an average of the fermentation tanks considering the value of multiple fermentation parameters relevant to productivity.

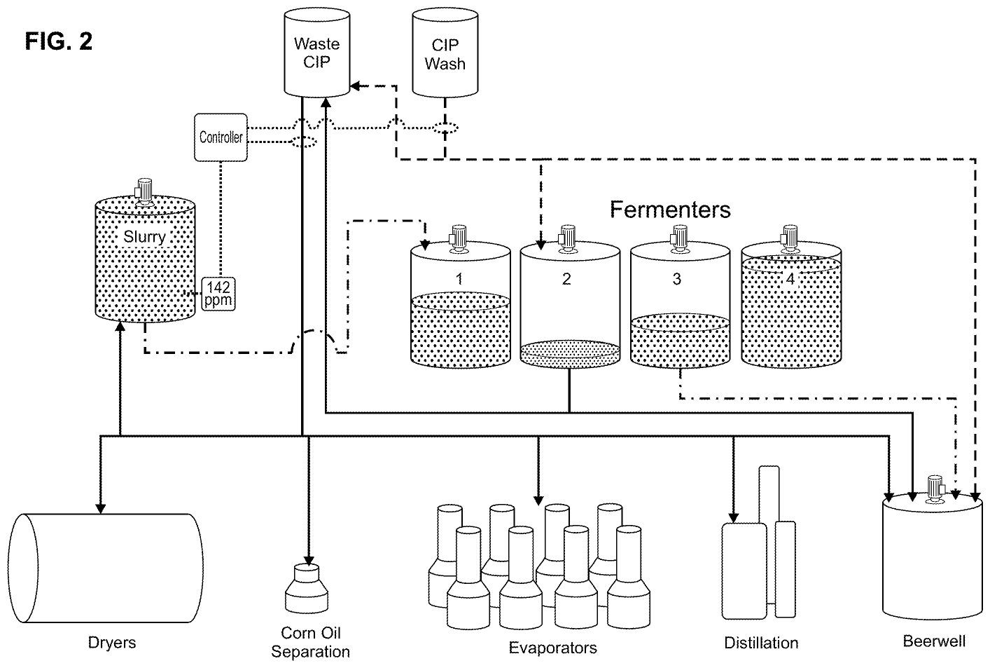

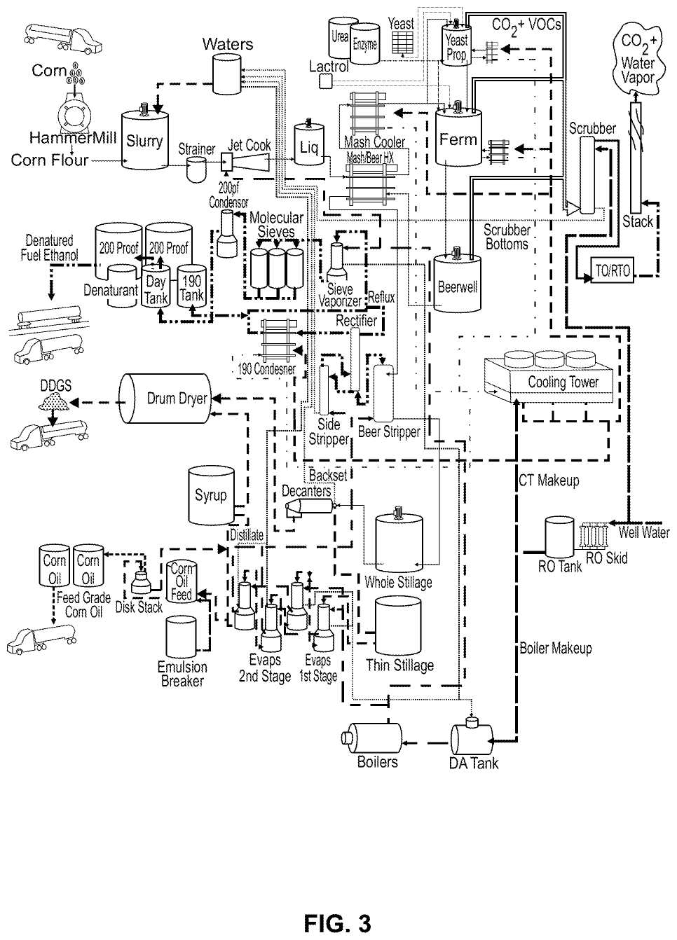

FIGS. 1-3 provide a generic scheme for a clean-in-place system as may be incorporated within an industrial fermentation system and process. As shown in FIG. 1, clean-in-place systems can include one or more fermentation tanks which are fed by pre-fermentation reagent streams. The fermentation tank is shown as one tank in FIG. 1 but it is understood that plants typically have from 1-30, from 2-15, or from 3-8 fermentation tanks. As discussed herein, pre-fermentation operations can include slurry and liquefaction tanks generally processing corn into corn mash and providing to the fermentation tanks. Pre-fermentation operations also can include yeast propagation tanks and stores of nutrients, additives, and supplements to promote yeast generation, start the yeast activity (e.g., reduce lag time), and promote fermentation within the fermentation tank. After fermentation, the product streams leave the fermentation tanks to various post-fermentation processing equipment, as shown and labeled in FIGS. 2-3. Post-fermentation processing provides solid outlets via the dryer and distiller's dried grains and solubles (DDGS), syrup, corn oil, and recycled waters to be reintroduced into the slurry.

Clean-in-place systems disclosed herein may generally be arranged at any point within the fermentation system. In certain aspects, clean-in-place systems can interface directly with one or more fermentation tanks, such that clean-in-place solutions solutions can be directed to the fermentation tanks independently from one another. Clean-in-place systems also can interface with the beerwell in order to access the post-fermentation process. This allows used clean-in-place solutions to be bled off into the post-fermentation process and not directly into the fermentation tanks. As shown in FIG. 1, it is also contemplated herein that clean-in-place systems may be incorporated into fermentation systems within lines running from the fermentation tanks to the beerwell such that clean-in-place solutions may alternately be applied to the fermentation tank or the beerwell depending on the operation state of the fermentation system, or user preference.

Clean-in-place systems of FIG. 1 are shown comprising separate tanks for storing and providing CIP chemicals to the fermentation system. Clean-in-place systems contemplated herein also can comprise an interface (not shown) allowing users to both receive and send information processed at a controller. The interface may be a screen monitor such as a touch screen and provide direct and real time feedback from the clean-in-place system generated from any parameters disclosed herein (e.g. sodium ion concentration within the fermentation tank, clean-in-place tanks, any recycle stream, or transfer lines), the sensors within the clean-in-place system and the fermentation system, and the fermentation system itself. The clean-in-place interface can display fermentation parameters as described herein. The clean-in-place interface also can provide a user control over the process systems via the clean-in-place controller.

The clean-in-place controller is not required to interact with any particular aspect of the clean-in-place system or fermentation system, however, it will be understood by those of skill in the art that the controller can operate to adjust flow rates from any or all of the clean-in-place tanks into the clean-in-place supply line. In certain embodiments, the clean-in-place controller may also operate to adjust flow rates into individual fermentation tanks of the fermentation system, for instance by closing appropriate valves in the clean-in-place supply line arrangement. In some embodiments, the clean-in-place controller is configured to operate the flow leaving any or all fermentation tanks into the clean-in-place return line. Again, though not necessarily pictured in the schematic representation of FIG. 1, those of skill in the art will understand junctions in fluid communication with the clean-in-place supply and clean-in-place return lines and other process stream lines can also be operated by the controller to ensure the flow is directed as intended. Alternatively, a separate fermentation controller can be provided to manage the clean-in-place system and each component unique to the fermentation system. In such embodiments, the clean-in-place controller and fermentation controller can be configured in direct communication to send, receive, and process data.

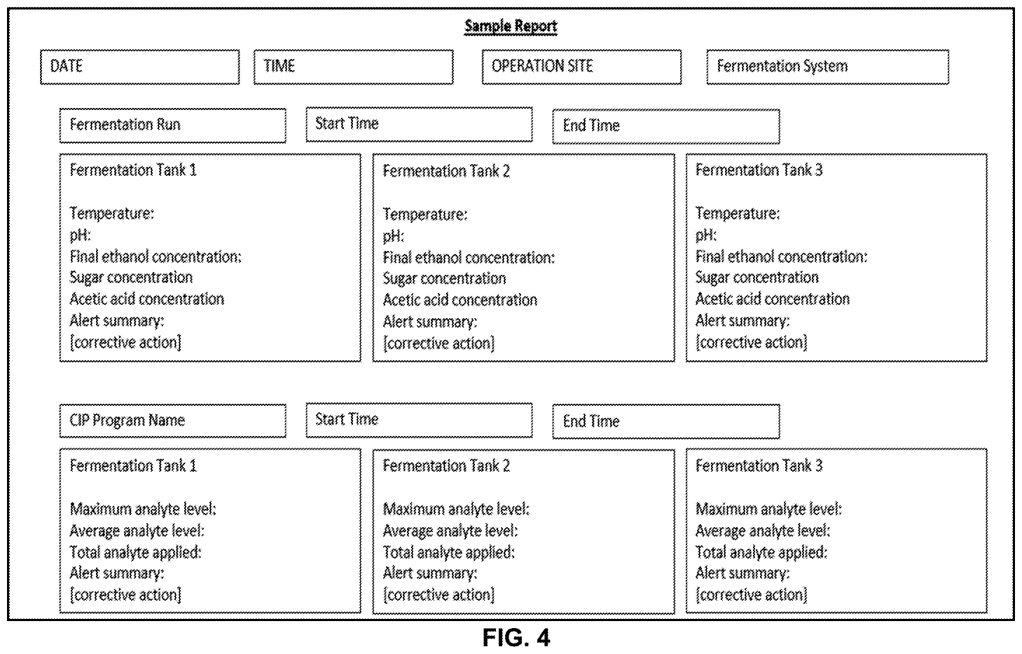

The clean-in-place controller also may be able to generate report data as described herein and format data as output to the user. In certain aspects the report data generated may be communicated to the clean-in-place interface and displayed as a digital and real-time report. Alternatively, or additionally, the report data may be iterated into a stored digital file that can be accessed on-demand, remotely, from a central database, or any combination thereof. The clean-in-place controller also may generate physical reports conveying the report data. FIG. 4 provides an example of such a report comprising certain systems data as described herein.

### EXAMPLE 1

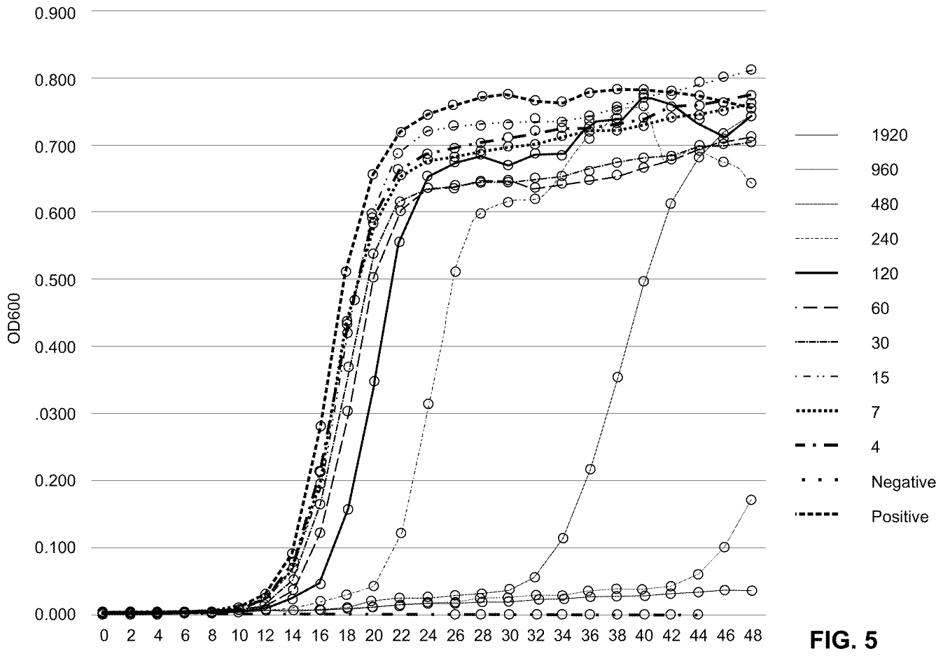

Sodium sensitivity of fermentation processes at each of 11 sodium levels and for a negative and positive control as shown in FIG. 5. Yeast was allowed to propagate over a period of 24 hours at similar conditions, and the optical density of each sample was recorded every two hours. As shown, levels of sodium in excess of 60-120 ppm observed a significantly delayed startup relative to higher sodium levels, and on the order of 4-8 hours before approaching maximum growth.

### Prophectic Example

Based on the results of Example 1 above, fermentation runs are contemplated for each of a high sodium and low sodium fermentation run. Each fermentation can be performed in a fermentation plant comprising six fermenters. In the high sodium fermentation, sodium levels were maintained at 120 ppm, whereas sodium levels in the low sodium fermentation were maintained below 60 ppm throughout the fermentation. Each fermentation was carried out for a period of 3 days, and conditions were maintained equivalent other than sodium levels.

Based on the data in FIG. 5, the yeast startup phase of the high sodium fermentation would be delayed by approximately 3 hours relative to the low sodium fermentation, approaching a maximum production after 20 hours and 17 hours respectively. Over the three-day fermentation runs, the low sodium fermentation can be considered operating at maximal production for a period of 55 hours, whereas the high sodium fermentation may only operate for 52 hours near maximum capacity, or an increase of more than 5%. Accordingly, it is contemplated that the high sodium fermentation may produce 105,000 gallons of 200 proof ethanol during the fermentation, whereas the low sodium fermentation may produce in excess of 108,750 gallons. Repeating this margin over the course of a year for a fermentation plant could generate additional millions of dollars in ethanol production.

### EXAMPLE 2

Experiments were performed to evaluate the removal of a caustic during the cleaning process. Melamine tiles with baked on starch were soaked in 1-liter beakers including buffering solutions with varying reagent concentrations. The buffering solutions included 998 grams of deionized water, 50 ppm of citric acid, and 50 ppm of calcium chloride. Some beakers of buffering solution also included 0 ppm to 1000 ppm of potassium chloride and 0 ppm to 1000 ppm of sodium chloride.

Citric acid acts as a buffering agent to maintain a stable pH of 4.0. Calcium chloride represents the calcium that is brought into a fermentation tank with corn in the fermentation process. Potassium chloride represents the potassium that is brought into a fermentation tank with corn in the fermentation process. Sodium chloride is used to evaluate the impact of sodium on enzyme cleaning performance. The pH of the solution was adjusted to 4.0 using potassium hydroxide (KOH) and hydrochloric acid (HCL).

Multiple beakers were prepared according to the reagents above with varying concentrations of potassium and sodium. The beakers were placed into a water bath at 41° C. Coupons were cut into 3 cm by 10 cm strips and the initial L reading was measured using a Mach 5 Image Analysis Spectrophotometer. The L reading is an indicator of intensity of reflected light of a specific wavelength on a bright coupon. A higher L value indicates more soil removal, a lower L value indicates more soil.

Enzyme performance for two commercially available blends of amylase enzymes (Amylase Blend #1 and Amylase Blend #2) used in the corn ethanol industry was tested in the presence of potassium and sodium salts.

The enzymes were added to beakers in quantities of 10 ppm for Amylase Blend #1 or 25 ppm for Amylase Blend #2, respectively. The cut coupons were placed into the beakers with varying reagent and enzyme concentrations. Coupons were soaked in beakers containing Amylase Blend #1 for 30 minutes, and a coupon was removed every 7.5 minutes for testing. Coupons were soaked in beakers containing Amylase Blend #2 for 120 minutes, and a coupon was removed every 30 minutes for testing. The coupons were dried overnight before the final L value was measured in the Mach 5 spectrophotometer.

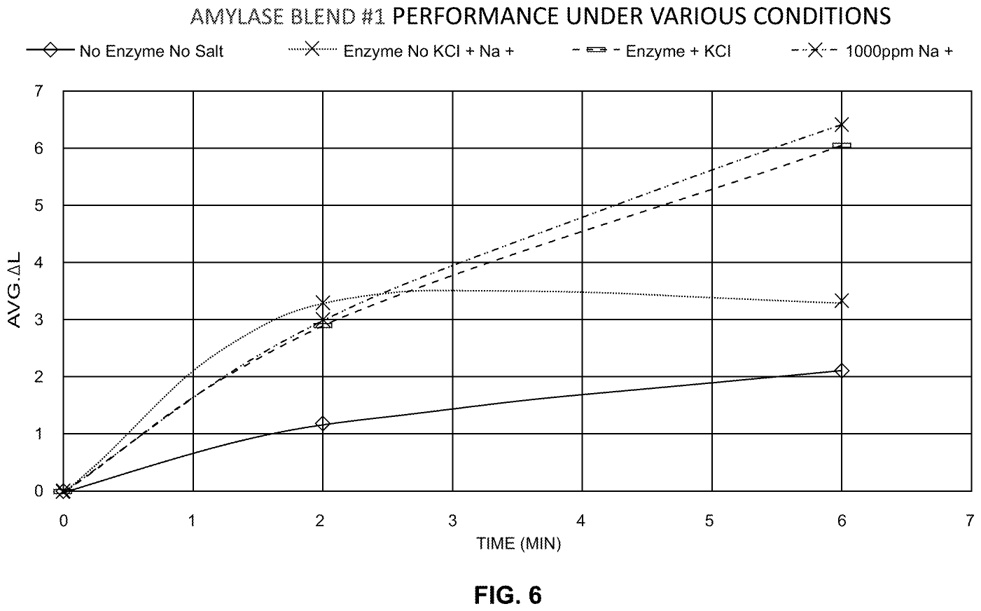

The activity of each enzyme was measured with varying concentrations of potassium and sodium. FIG. 6 shows the performance of Amylase Blend #1 under various conditions. The four lines represent a control measurement with no enzyme and no salt (i.e., no potassium or sodium), enzyme and no salt, enzyme with potassium, and 1000 ppm sodium. The enzyme performance was increased with the addition of potassium chloride as compared to performance of the enzyme with no salts. Adding sodium to the enzyme, however, did not result in a significant difference compared to the enzyme with potassium.

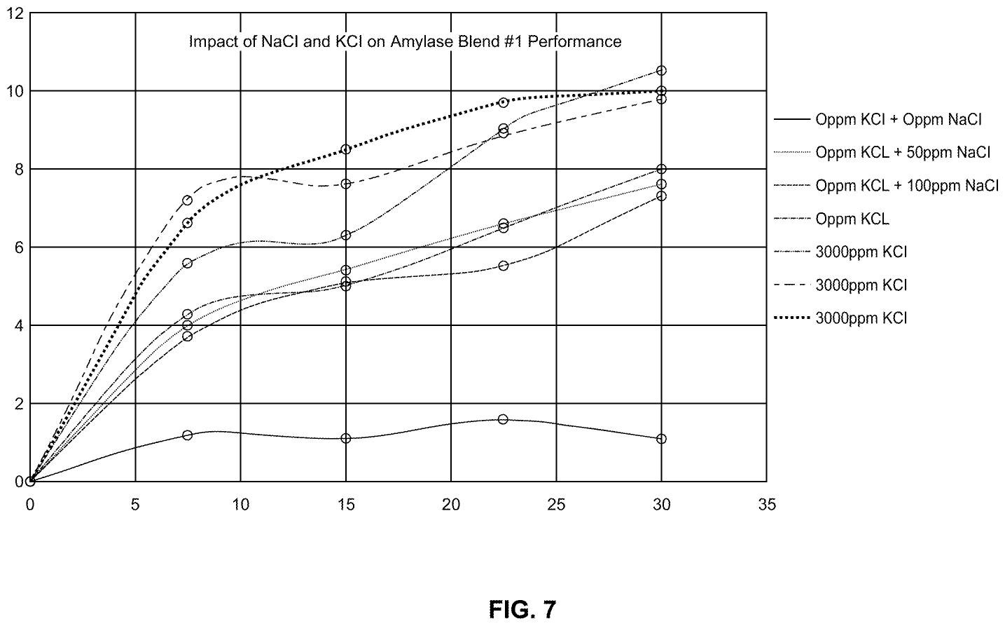

Amylase Blend #1 performance was also measured over the course of a 30 minute test period using the coupons soaked in beakers with various reagents and concentrations. FIG. 7 shows the results of Amylase Blend #1 performance with and without KCl and NaCl at various concentrations. Enzyme performance was highest in the presence of potassium. The presence of sodium does not impact the performance of Amylase Blend #1 as much as potassium does.

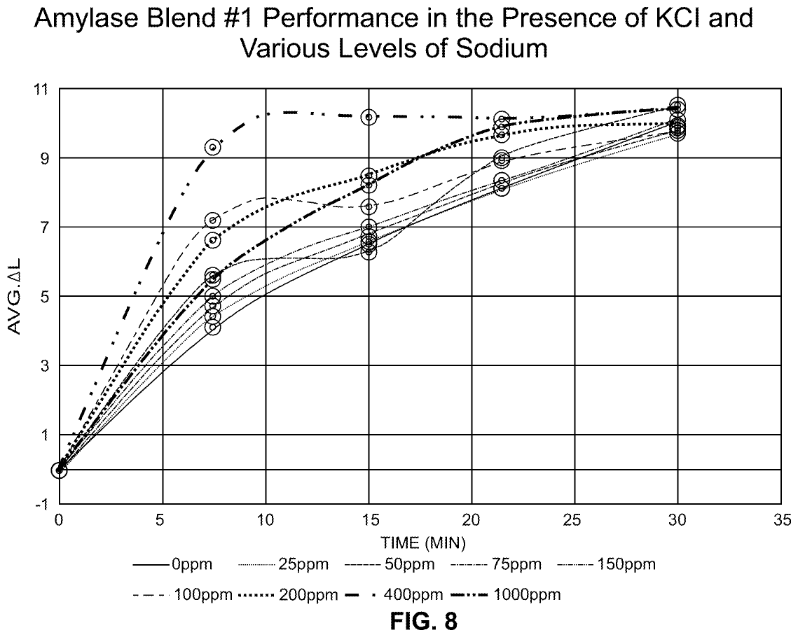

FIG. 8 illustrates how various concentrations of sodium impacts the performance of Amylase Blend #1 with a constant concentration of potassium. In each experiment, 0-1000 ppm of sodium was added to 3000 ppm of potassium and Amylase Blend #1 to evaluate performance over 30 minutes. The addition of increasing sodium concentrations increased performance of Amylase Blend #1 with potassium up to 400 ppm sodium. 1000 ppm of sodium did not increase enzyme activity as much as 400 ppm sodium did, but 1000 ppm sodium still increased enzyme activity above the activity of only potassium and the enzyme.

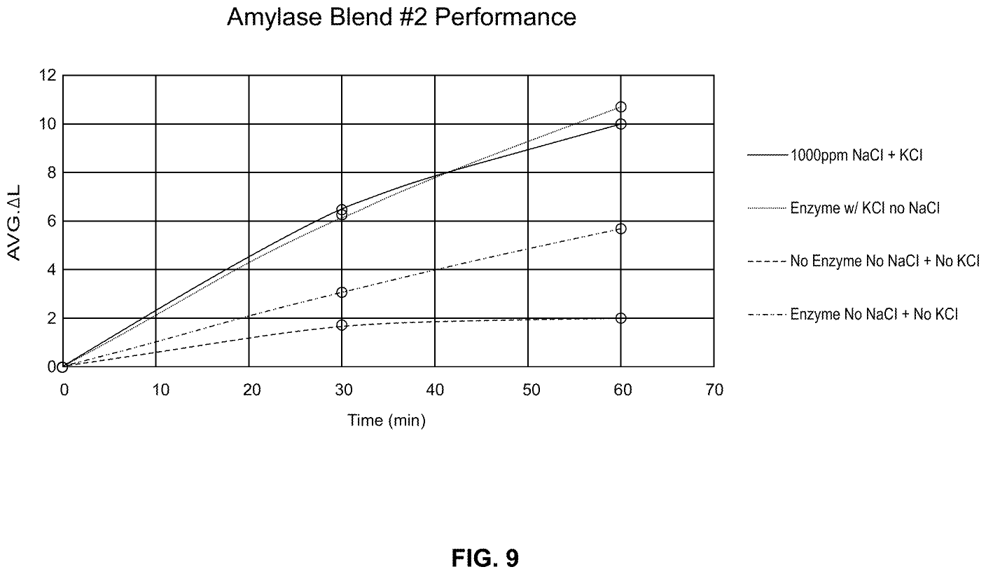

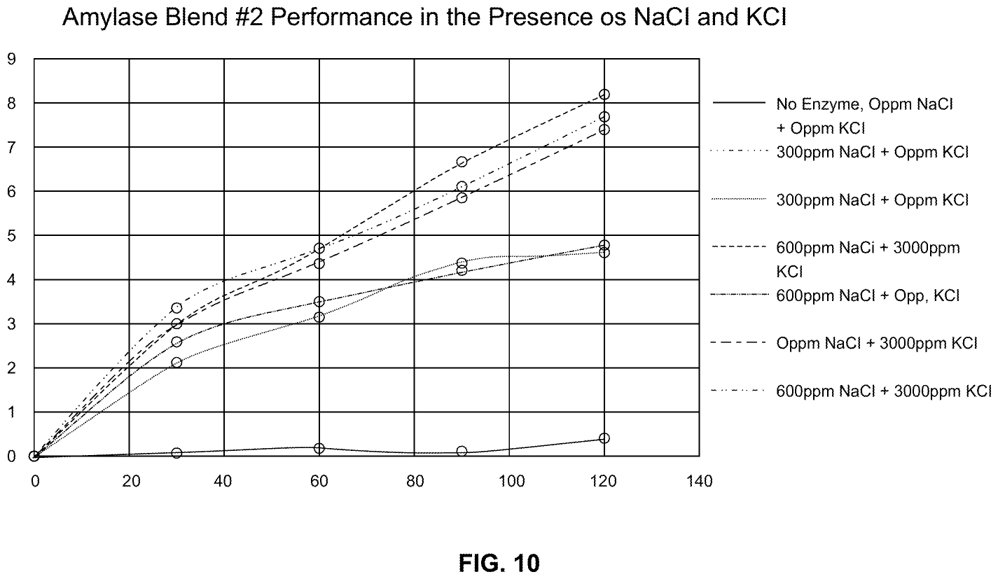

Amylase Blend #2 activity was measured without any salts, with KCl, and with both NaCl and KCl. FIG. 9 shows that the enzyme activity is highest in the presence of potassium and that there is minimal impact with the presence of sodium in addition to potassium. Amylase Blend #2 performance was also measured over the course of 120 minutes to evaluate the effects of sodium and potassium on the enzyme over time, as shown in FIG. 10. The addition of only NaCl to the enzyme slightly increased enzyme performance, but the addition of KCl, alone or in combination with NaCl greatly increased the performance of the enzyme.

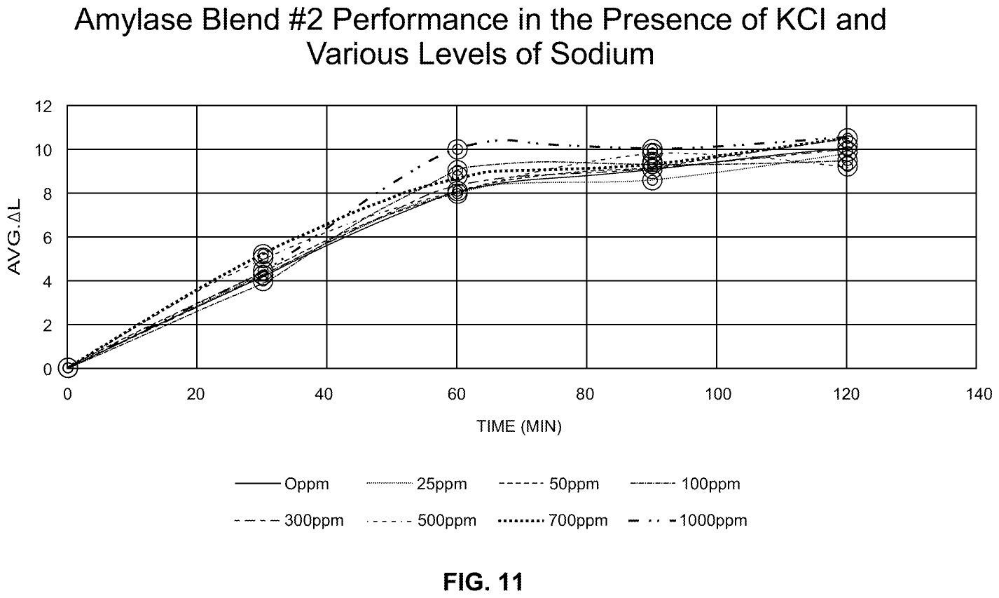

FIG. 11 illustrates how various concentrations of sodium impact the performance of Amylase Blend #2 with a constant potassium concentration of 3000 ppm over 120 minutes. Ranges of sodium from 0 ppm to 1000 pm increased enzyme performance at some time points, but sodium demonstrated a lesser effect on enzyme performance for Amylase Blend #2 as compared to Amylase Blend #1.

Performance of Amylase Blend #1 or Amylase Blend #2 without salts demonstrated lower activity, while enzyme performance was strongest with KCl, and sodium alone did not meaningfully increase enzyme activity. Either enzyme with only potassium performed similarly to the combination of enzyme, potassium, and sodium. Thus, sodium does not play a key role in the performance of Amylase Blend #1 and Amylase Blend #2. Potassium that is part of corn in the fermentation system drives enzyme activity, and sodium can be omitted or removed without impacting enzyme performance.

The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A method of managing the clean-in-place of a fermentation operation while improving fermentation efficiency comprising:
a. monitoring a fermentation parameter;
b. determining if the fermentation parameter is within an acceptable range; and
c. modifying a clean-in-place program to bring the fermentation parameter within the acceptable range.

2. The method of claim 1, wherein the fermentation parameter is selected from the group of sodium ion concentration, potassium ion concentration, acetic acid concentration, lactic acid concentration, urea concentration, ammonia concentration, sugar concentration, ethanol concentration, an antibiotic concentration, a sulfuric acid concentration, temperature, pH, and combinations thereof.

3. The method of claim 2, wherein the acceptable range for sodium ion concentration is from 20 to 50 ppm sodium ion.

4. The method of claim 2, wherein the acceptable range for potassium ion concentration is from 500 to 5000 ppm potassium ion.

5. The method of claim 2, wherein the acceptable range for acetic acid concentration is below 0.1% acetic acid.

6. The method of claim 2, wherein the acceptable range for lactic acid concentration is below 0.8% lactic acid.

7. The method of claim 2, wherein the acceptable range for urea concentration is from 5 to 1000 ppm urea.

8. The method of claim 2, wherein the acceptable range for total sugars concentration is less than about 1%.

9. The method of claim 2, wherein the acceptable range for temperature is from 20 to 40° C.

10. The method of claim 2, wherein the acceptable range for pH is from 4.5 to 5.

11. The method of claim 2, wherein the acceptable range for pH is higher than the pKa of acetic acid.

12. The method of claim 2, wherein the acceptable range for pH is higher than the pKa of lactic acid.

13. The method of claim 1, wherein modifying the clean-in-place program comprises changing the concentration of a clean-in-place solution.

14. The method of claim 1, wherein modifying the clean-in-place program comprises replacing the clean-in-place solutions.

15. The method of claim 1, wherein modifying the clean-in-place program comprises adding a supplement to the clean-in-place program.

16. The method of claim 15, wherein the supplement is selected from the group of sodium ions, potassium ions, ammonium ions

17. The method of claim 16, wherein the supplements are sodium ions, potassium ions, or a combination of sodium and potassium.

18. The method of claim 17, wherein supplements maintain a ratio in the fermentation of tanks of potassium to sodium of about 10:1 to about 100:1.

19. The method of claim 14, wherein the clean-in-place solutions are replaced with chemicals that is free from sodium ions.

20. The method of claim 14, wherein the clean-in-place solutions are replaced with solutions that have a pH of less than 8.

21. The method of claim 1, wherein the fermentation operation comprises 3-20 fermentation tanks and the method further comprises adjusting the fermentation parameter to be +/−10% in all the fermentation tanks.

22. The method of claim 1, wherein modifying the clean-in-place program results in at least a 2% increase in ethanol production.

23. The method of claim 1, wherein modifying the clean-in-place program results in at least a 20% shorter lag time in yeast activity.

24. The method of claim 1, wherein modifying the clean-in-place program results in improved fermentation enzyme efficiency.

25. The method of claim 1, wherein modifying the clean-in-place program results in lower concentrations of acetic acid-generating or lactic acid-generating bacteria.

26. The method of claim 1, wherein modifying the clean-in-place program results in lower acetic acid or lactic acid concentrations in the fermentation operation.

27. A method of managing the clean-in-place of a fermentation operation while improving fermentation efficiency comprising:
a. monitoring a fermentation parameter in a plurality of fermentation tanks comprising corn mash;
b. determining if the fermentation parameter is within an acceptable range for each fermentation tank; and
c. blending corn mash between fermentation tanks so that the fermentation parameter for each fermentation tank is within +/−10%.

28. The method of claim 27, wherein the fermentation parameter is selected from the group of sodium ion concentration, potassium ion concentration, acetic acid concentration, lactic acid concentration, urea concentration, ammonia concentration, sugar concentration, ethanol concentration, temperature, pH, and combinations thereof.

29. The method of claim 28, wherein the acceptable range for sodium ion concentration is from 20 to 100 ppm sodium ion.

30. The method of claim 28, wherein the acceptable range for potassium ion concentration is from 500 to 5000 ppm potassium ion.

31. The method of claim 28, wherein the acceptable range for acetic acid concentration is below 0.1% acetic acid.

32. The method of claim 28, wherein the acceptable range for lactic acid concentration is below 0.8% lactic acid.

33. The method of claim 28, wherein the acceptable range for urea concentration is from 5 to 1000 ppm urea.

34. The method of claim 28, wherein the acceptable range for total sugars concentration is less than about 1%.

35. The method of claim 28, wherein the acceptable range for temperature is from 20 to 40° C.

36. The method of claim 28, wherein the acceptable range for pH is from 4.5 to 5.

37. The method of claim 28, wherein the pH is higher than the pKa of acetic acid.

38. The method of claim 28, wherein the pH is higher than the pKa of lactic acid.

39. The method of claim 27, further comprising adding a supplement to the clean-in-place program.

40. The method of claim 39, wherein the supplement is selected from the group of sodium ions, potassium ions, ammonium bisulfate, or a combination thereof.

41. The method of claim 39, wherein supplements maintain a ratio in the fermentation of tanks of potassium to sodium of about 10:1 to about 100:1.

42. The method of claim 27, wherein the fermentation operation comprises 3-20 fermentation tanks.

43. The method of claim 42, wherein the fermentation parameter is +/−5% in all the fermentation tanks.

44. The method of claim 27, wherein the method results in a 5% increase in ethanol production.

45. The method of claim 27, wherein the method results in a 5% shorter lag time in yeast activity.

46. The method of claim 27, wherein the method results in improved fermentation enzyme efficiency.

47. The method of claim 27, wherein the method results in lower concentrations of acetic acid-generating or lactic acid-generating bacteria.

48. The method of claim 27, wherein the method results in lower acetic acid or lactic acid concentrations in the fermentation operation.

49. The method of claim 27, further comprising recording the fermentation parameter.

50. The method of claim 49, wherein the recording is paper or electronic.

51. The method of claim 49, wherein the recording is done in a mobile device, in-line instrument, or software program.

52. The method of claim 51, wherein the mobile device or software program comprises data fields for date, time, location, site, fermentation tank, fermentation parameter, corrective action, ethanol yield, yeast health, enzyme health, or a combination thereof.

53. The method of claim 27, further comprising generating a report.

54. The method of claim 27, further comprising generating an alert if the fermentation parameter is not within an acceptable range.

55. The method of claim 54, wherein the alert is an email, mobile phone message, visual, or audio alert.

56. The method of claim 54, wherein the alert is noted on a report.
